(19) United States
(12) Patent Application Publication
(10) Pub. No.: US 2013/0116471 A1
(43) Pub. Date: May 9, 2013

(54) PROCESS FOR PREPARING LONG-CHAIN DICARBOXYLIC ACIDS AND THE PRODUCTION THEREOF

(71) Applicants: LIMING YAN, Beijing (CN); Yong Yang, Beijing (CN); Shenzhan Fu, Beijing (CN); Yuantong Chen, Beijing (CN); Zhizhou Wang, Beijing (CN); Wubo Cao, Beijing (CN)

(72) Inventors: LIMING YAN, Beijing (CN); Yong Yang, Beijing (CN); Shenzhan Fu, Beijing (CN); Yuantong Chen, Beijing (CN); Zhizhou Wang, Beijing (CN); Wubo Cao, Beijing (CN)

(73) Assignees: SHANDONG HILEAD BIOTECHNOLOGY CO., LTD., Laiyang (CN); INSTITUTE OF MICROBIOLOGY CHINESE ACADEMY OF SCIENCES, Beijing (CN)

(21) Appl. No.: 13/456,926

(22) Filed: Apr. 26, 2012

(30) Foreign Application Priority Data
Sep. 30, 2011 (CN) 201110303844.X

(51) Int. Cl. C07C 51/43

(52) U.S. Cl. CPC C07C 51/43
USPC 562/593; 562/590

(57) ABSTRACT

An organic solvent free method for purifying and refining of a long-chain dicarboxylic acid or a salt thereof is disclosed. This method avoids problems caused by organic solvents which have been used in the purifying process of the prior art. This method reduces effectively the content of such impurities as proteins and coloring materials in the product. The purity of the crystallized long-chain dicarboxylic acid product is greater than 99 wt %.

### FIELD OF THE INVENTION

The present invention relates to a process for purifying long-chain dicarboxylic acids and the production thereof, especially relates to a process for purifying long-chain dicarboxylic acids without using any organic solvents and the production thereof

### BACKGROUND OF THE INVENTION

Long-chain dicarboxylic acids are used as important raw material for synthesizing such products as Nylon, high-grade perfume, plasticizers, lubricants, as well as advanced thermosols. Nowadays various types of long-chain dicarboxylic acids are mainly produced as metabolic products is obtained from the fermentation with n-alkane by microorganisms such as Candida tropicalis. However the fermentation liquid is a complex multi-phase system which contains unreacted n-alkane, unutilized culture medium, cells of microorganisms and inclusions thereof, secretion substances of the microorganism and the like, especially in which a large amount of impurities, such as proteins and pigments, will have serious effect on the purity and appearance of the long-chain dicarboxylic acid the product.

Currently, the process for purifying the long-chain dicarboxylic acid is typically classified into two types: a process using organic solvents and a process using water. The process using organic solvents is significantly restricted by the problems such as high investment, residual alkane and solvent in the product, safety of production and so on. The process using water can overcome the defects that the solvent process has, however, the purity of its product using the current techniques cannot attain to a high level required by polymerization. For example, JP56026194 disclose an aqueous phase process for separating dicarboxylic acid, wherein the purification steps comprise alkalifying and stewing the fermentation liquid, centrifuging to remove microorganism, adding siliceous earth to adsorb unreacted reactants and by-products, then filtering, acidifying and depositing the filtrate. Finally, the product of dicarboxylic acid is obtained after filtration and drying. The problem existing in the processes mentioned above is the autolysis of cells during alkalifying and stewing, thereby the impurities such as proteins and coloring materials in cells are dissolved into the fermentation liquid, consequently the purity of total acid product is only 98.5% at highest, and the coloring materials in the product are difficult to be removed. The product is then light brown in appearance. Moreover, the main problem of the aqueous phase process is that monocarboxylic acid sodium salt is co-crystallized with dicarboxylic is acid monosodium salt or disodium salt. Thus it is hard to separate the dicarboxylic acid from similar monocarboxylic salt thereof.

CN1255483A disclosed a method for purifying long-chain dicarboxylic acid product by using crystallization of the monosalt of long-chain dicarboxylic acid. This method needs to precisely adjust the amount of base added in the fermentation liquid to make sure the dicarboxylic acid generating only the monosalt and avoiding to become the disodium salt. However it is hard to decide the exact amount of base when the mol. production ratio of the dicarboxylic acid is hard to predict. This defect leads to the yielding ratio of the product is rather low to about less than 90 wt % without recycling the dicarboxylic acid in the filter of the monosalt. And the yielding ratio of the product could reach to about 92 wt % with recycling the dicarboxylic acid in the filter of the monosalt, which however increases the processing costs. And the aqueous phase of the monosalt filter is not pure enough, and it is hard to remove the residual monosalt in the final production. CN1219530A disclosed a method for purifying long-chain dicarboxylic acid product by salting out with disodium salt. However the product ratio is still low and the filtration of the salting out also has to recycle to obtain the dicarboxylic acid.

CN1552687A disclosed a process for refining a long-chain dicarboxylic acid product by heating it to the melting points, and followed by addition of water to carry out cooling crystallization. After reaching the melting point, dicarboxylic acid which is floating on the surface of aqueous phase has been collected in the open environment. As melting points of most dicarboxylic acids with even number of carbons are above 110 degree Celsius, particular methods such as using highly concentrated saline to increase the water boiling temperature has been described in the patent. However such adjustment of the method confines is its application. Moreover, the separation of the dicarboxylic acid in the melting state has been proved very difficult: there are no obvious floating layers of the DC₁₀, DC₁₂ and DC₁₃. In addition, crystallization by adding water causes precipitation of both monocarboxylic and dicarboxylic acids. The crystals and the amorphous precipitates are also mixed together, which makes separation of the crystalised dicarboxylic acid difficult. Such method may solve the purification of the DC₅, DC₇ and DC₉, but it is very difficult to use such method to deal with the purification of the other dicarboxylic acid with even carbon number.

### SUMMARY OF THE INVENTION

The purpose of the present invention is to provide a purifying and refining method for all the dicarboxylic acids by aqueous crystal without using any types of saline or organic solvents, and with no need to separate the dicarboxylic acid layer from the aqueous surface. More specifically, the method for purifying long-chain dicarboxylic acids includes the following steps:

1) . Acidifying a liquid containing a long-chain dicarboxylic acid and/or a salt thereof, and collecting a crude product of the dicarboxylic acid which is in the crystal state or in the amorphous state;

2) . Dissolving the crude product of the dicarboxylic acids using an alkaline solution with heating to obtain a solution, and filtering the solution to remove an insoluble material in the solution and decoloring with a decoloring agents to obtain a filtered solution;

3) . Acidifying the filtered solution to adjust the pH value of the fermentation liquid to a range of 1˜2.5, then collecting a deposit of the dicarboxylic acid after centrifuging the filtered solution;

4). Washing the deposit of the dicarboxylic acid to neutrality by adding water with a mass of 3 to 20 folds of that of the dicarboxylic acid is to re-suspend the dicarboxylic acid, heating the deposit of the dicarboxylic acid up to 60˜100° C., filtering, washing, and drying the deposit of the dicarboxylic acid to get a filtration cake of the dicarboxylic acid;

5). Suspending the filtration cake of the dicarboxylic acid in water and heating a mixture of the filtration cake of the dicarboxylic acid and water up to a temperature above 100° C. under high pressure, and maintaining the temperature above a melting point of the dicarboxylic acid for 20˜30 minutes,

6). Slowly dropping the temperature of the filtration cake of the dicarboxylic acid and water to a temperature in range of 25˜30° C. and procuring the dicarboxylic acid crystals by filtration.

Preferably, in the process 1) of the present invention, the pH value during the step of acidifying and depositing the filtrate is in a range of 1˜2.5, a temperature thereof is 60˜100° C.

Preferably, in the process 2) of the present invention, 5M NaOH is used as the alkaline solution to heat and melt the dicarboxylic acid, and the dealing condition is at 80° C. for one hour.

Preferably, in the method of filtering to remove the insoluble material of the process 2) of the present invention, the filter medium includes one kind or several kinds of gauze, ceramic film, metal film or glass fiber membrane is used.

Preferably, in the process 2) of the present invention, the decoloring agent used for decoloring is an activated carbon with 0.2% w or a siliceous earth with 0.5% w.

Preferably, in the process 4) of the present invention, the deposit of the dicarboxylic acid is wasted till a pH value of the dicarboxylic acid is in range of 6.5˜7.0.

Preferably, in the process 4) of the present invention, the mass of is said water used in washing the deposition of the dicarboxylic acid is 5 folds to that of the dicarboxylic acid.

Preferably, in the process 4) of the present invention, said water used in washing the deposition of the dicarboxylic acid is at 85° C. and with neutrality pH value, and the deposit of the dicarboxylic acid is washed 1-10 times repeatedly.

Preferably, in the process 5) of the present invention, said temperature is equal to the melting point of the dicarboxylic acid, and the duration of the heating is 10˜60 minutes.

Preferably, in the process 6) of the present invention, said temperature dropping process is to drop the temperature 10˜15° C. per hour.

Another purpose of the present invention is to provide dicarboxylic acids produced by using the method of anyone of the claims 1-11, wherein no organic solvent is used in the purification process.

The above mentioned method could also be applied to organic solvent solution and solid crude product which contains dicarboxylic acid and/or a salt thereof In the cases of organic solvent solutions, removing any organic solvent then processing the above step 2); In the cases of solid crude product; starting from the above step 2); If starting from an aqueous solution containing a long-chain dicarboxylic acid and/or a salt thereof, starting from the above step 1).

Technical Solution:

The method of the present invention is much easier than the current application using organic solvent such as acetic acid. Therefore, the production ratio is much higher. Moreover, the water used in the crystallization could be used for washing the filtering cake and recycling. As without using acetic acid and with no acetic acid discharge, the method is much more advantaged for environmental protection. Finally, by using the method of falling down the temperature and filtering with water solution can acquire the dicarboxylic acid with higher purity up to 98.6%, and the crystallization of the dicarboxylic acid which is in the specific state and different from former purification methods is acquired. The present method of the invention is particularly suitable for the raw material for synthesizing products such as high-grade perfume and advanced thermosol and so on.

### BRIEF DESCRIPTION OF THE FIGURES

FIG. 1A illustrates the crystals of the dodecanedicarboxylic acid produced by the method of the present invention;

FIG. 1B illustrates the crystals mixed with amorphous powder of the dodecanedicarboxylic acid produced by the method of the patent document CN102061316A (commercial product);

FIG. 2A illustrates the HPLC chromatogram of dicarboxylic acid crystallized using the method of present invention;

FIG. 2B illustrates the HPLC chromatogram of dicarboxylic acid crystallized using the method of the patent document CN102061316A (commercial product).

### DETAILED DESCRIPTION OF THE INVENTION

The strain used in the embodiment of the present invention is Candida tropicalis UH-2-48 (deposit number CGMCC 0239, disclosed in CN1130685A). The crude production of the dicarboxylic acid from the fermentation of other strains, or the crude production of the dicarboxylic acid from synthetic chemistry method could use the same purification method described in the present invention.

Raw materials for producing dicarboxylic acids could be selected from alkanes with different chain length, various fatty acids or their esters. Dodecane, and sodium palmitate are used in the embodiment of the present invention. Various raw materials could be used in producing dicarboxylic acids with different chain length. The final temperature for heating up the mixture of a dicarboxylic acid can be referred to the following table 1.

TABLE 1
Suggested heating temperatures of the mixture of various
dicarboxylic acids and water

| Number of | |
|---|---|
| carbon | |
| atoms | Heating temperature ° C. |
| 2 | 189-191 |
| 3 | 131-135 |
| 4 | 185-190 |
| 5 | 95-99 |
| 6 | 151-153 |
| 7 | 105-106 |
| 8 | 143-144 |
| 9 | 100-103 |
| 10 | 131-134 |
| 11 | 109-110 |
| 12 | 128-129 |
| 13 | 112-114 |
| 14 | 126-128 |
| 16 | 124-126 |
| 18 | 123-125 |

The present invention will be illustrated by the following examples.

### EXAMPLE 1

1. Culturing, seedling and fermentation of the strain for dodecanedicarboxylic acid production

The strain used in the embodiment of the present invention is Candida tropicalis UH-2-48(deposit number CGMCC 0239). A 50 ml starter culture is prepared from a conventional slant, and cultivated for 16 hours, followed by being transferred to a 1L secondary starter culture for another 16 hours. The starter culture contains corn syrup 0.2˜0.5% (w/v), yeast extract 0.3˜0.7% (w/v), urea 0.2˜0.5% (w/v), sucrose 2-5% (w/v), KH₂PO₄ 0.5˜1% (w/v), defoamer 0.03˜0.05% (v/v).

The aforementioned secondary starter culture is finally transferred to is a 10L fermenter tank to carry out the final fermentation. The basic media of the culture with a volume of 4L contains corn syrup 2-7% (w/v), NaCl 0.1%˜0.3% (w/v), yeast extract 0.15˜0.3% (w/v), urea 0.1˜0.25% (w/v), glucose 3˜7% (w/v), sucrose 0.5˜2% (w/v), KNO3 1˜2% (w/v), KH2PO4 0.5˜2% (w/v), cell regulators: 3˜7% (w/v), emulsifier 0.001˜0.05% (v/v), defoamer 0.03% (v/v). The fermentation is carried out for 144-156 h at 30° C. with a ventilation volume at 1:0.5. Dodecane is supplemented at the rate of 50 ml/h after the fermentation for 16 h till 150 g of dodecane (approximately 2L) is thoroughly supplemented. The pH value is controlled at 7.2 by 10M NaOH solution, and the dissolved oxygen is maintained at 20% through adjusting the rotational speed.

2. Procurement of the crude dodecanedicarboxylic acid

1) A 7L fermentation liquid, which is obtained in the above fermentation procedures with a final concentration of the total acid content at 187 g/l, is heated to 80° C. and maintained for 60 minutes.

2) After pH value of NaOH (10M) is adjusted to 9.5, the yeast cells are removed by centrifuging.

3) Active carbon powder at final amount of 0.5% (w/v) is added to the supernatant and the filtrate is incubated at 70° C., which is maintained for 60 minutes.

4) Active carbon is removed by vacuum filtration with a 0.44 μm nylon membrane;

5) The filtering solution is acidified to pH 2.5 by using 98% sulfuric acid, and incubated the filtering solution at 80° C. for 2 hours.

6) The crude dodecanedicarboxylic acid is obtained by centrifuging the precipitate formed at the step 5). The precipitation is then washed by water and re-suspended, and then the precipitation is centrifuged, followed by vacuum drying to obtain the crude dodecanedicarboxylic acid.

3. The pretreatment process before crystallization

1) The crude dodecanedicarboxylic acid is re-dissolved by using 5M NaOH solution with a proportion of 1.74 g/ml, which is then heated to 80° C. and maintained for one hour. The solution containing re-dissolved dodecanedicarboxylic acid is then filtered to remove insoluble impurities with a piece of filter paper at 80° C. in a cabinet drier.

2) Active carbon powder at a final amount of 0.2% (w/v) or siliceous earth at 0.5% (w/v) is then added to the filtrate, which is carried out at 60° C. and incubated at the temperature for 60 minutes.

3) Active carbon or siliceous earth powder is removed by vacuum filtration with a 0.44 μm nylon membrane;

4) The filtered-through solution is acidified to or lower than pH 2.5 using 98% sulfuric acid so that dodecanedicarboxylic acid is precipitated from the solution;

5) The deposit of dodecanedicarboxylic acid is collected by centrifuging;

6) The obtained dodecanedicarboxylic acid is then re-suspended and washed by using water with three-fold of the acid mass, followed by centrifuging. This washing step is repeated till the pH value of the washing supernatant is higher than 6.5. This washing step is usually carried out for three times. Water with mass of five-folds of that of the dicarboxylic acid is added and the suspension is heated to 80° C., is followed by filtration to get the filtered cake. The cake is then washed for three times using water at 85° C.

4. The crystallization process of the dodecanedicarboxylic acid

1) The filtering cake of the washed dodecanedicarboxylic acid is re-suspended in water with mass of five-folds of that of the filtering cake. The mixture is then heated to 128˜130° C. using a pressure vessel(GSHA-2 (2L), Henghua Chemical Plant, Weihai, P.R. China), and maintained the temperature for 20˜30 minutes.

2) The temperature of the mixture is dropped continuously and slowly to the room temperature at a rate of 10° C. per hour. Dodecanedicarboxylic acid will be crystallized during the temerature dropping process;

3) The crystallized dodecanedicarboxylic acid is obtained by centrifuging. Some float on the aqueous surface of the mixture, which contains monocarboxylic acid, is separated and removed using this method;

4) The residual moisture is dried in a vacuum oven; and the purified dodecanedicarboxylic acid is procured.

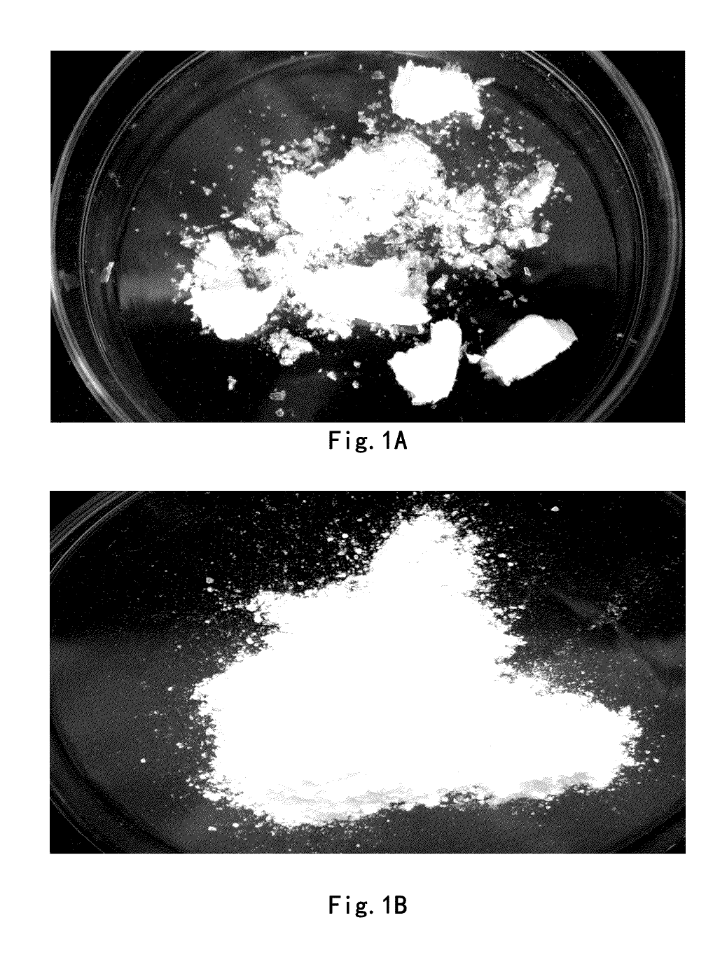

The obtained dodecanedicarboxylic acid is 138.2 g in total, and the purity is 99.2%, the yield of product is 95.2%. The final product of dodecanedicarboxylic acid using the method of this example 1 showed crystal appearance (FIG. 1A), and that using the method of the example 1 of CN 102061316A showed amorphous appearance (FIG. 1B). The final product of dodecanedicarboxylic acid using the present invention is particularly suitable for downstream polymer synthesis such as polyamides, which requires highly pure precursors.

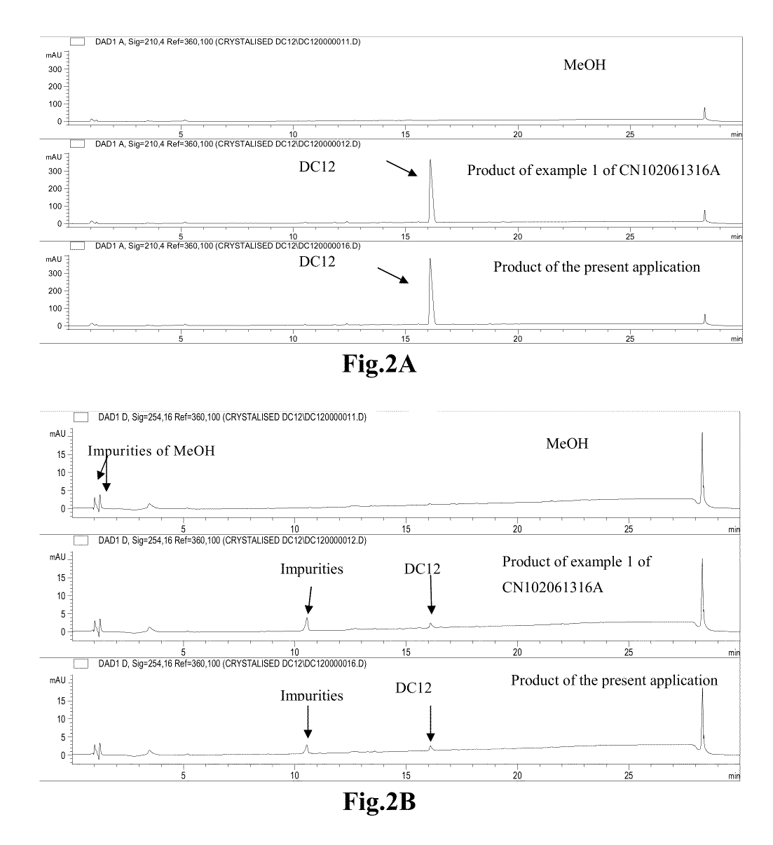

FIG. 2A illustrates the HPLC chromatogram at 210 nm and FIG. 2B at 254 nm. The condition of HPLC is using Agilent 1260, and the column is Zorbax Eclipse plus C18, the Diode Array Detector is G1315D. The mobile phase is 50% water: 50% acetonitrile at the beginning, and the end is 100% acetonitrile, and eluting in 30 minutes with the flow velocity of 1 ml/min. It can be concluded from the FIG. 2.A and FIG. 2.B that the purification and refinery process applied in the present patent can procure a product with the same quality as the current industrial product, even with less impurities.

### EXAMPLE 2

1. Culturing, seedling and fermentation of the strain for tridecanedicarboxylic acid production

The strain used in the embodiment of the present invention is Candida tropicalis UH-2-48(deposit number CGMCC 0239). A 50 ml starter culture is prepared from a conventional slant, and cultivated for 16 hours, followed by being transferred to a 1L further secondary starter culture for another 16 hours. The starter culture contains corn syrup 0.2˜0.5% (w/v), yeast extract 0.3˜0.7% (w/v), urea 0.2˜0.5% (w/v), sucrose 2˜5% (w/v), KH₂PO₄ 0.5˜1% (w/v), defoamer 0.03˜0.05% (v/v).

The aforementioned secondary starter culture is finally transferred to a 10L fermenter tank to carry out the final fermentation. The basic media of the culture with a volume of 4L contains corn syrup 2˜7% (w/v), NaCl 0.1%˜0.3%(w/v), yeast extract 0.15˜0.3% (w/v), urea 0.1˜0.25% (w/v), glucose 3˜7% (w/v), sucrose 0.5˜2% (w/v), KNO3 1˜2% (w/v), KH2PO4 0.5˜2% (w/v), cell regulators: 3˜7%(w/v), emulsifier 0.001˜0.05%(v/v), defoamer 0.03% (v/v). The fermentation is carried out for 144-156 hours at 30° C. with a ventilation volume at 1:0.5. Tridecane is supplemented at the rate of 50 ml/h after the fermentation for 16 hours till 150 g of tridecane (approximately 2L) is thoroughly supplemented. The pH value is controlled at 7.2 by 10M NaOH solution, and the dissolved oxygen is maintained at 20% through adjusting the rotational speed.

2. Procurement of the crude tridecanedicarboxylic acid

1) A 7L fermentation liquid, which is obtained in the above fermentation procedures with a final concentration of the total acid content at 177 g/l, is heated to 80° C. and maintained for 60 minutes.

2) After pH value of NaOH(10M) is adjusted to 9.5, the yeast cells are removed by centrifuging.

3) Active carbon powder at final amount of 0.5% (w/v) is added to the supernatant and the filtrate is incubated at 70° C., which is maintained for 60 minutes.

4) Active carbon is removed by vacuum filtration with a 0.44 μm nylon membrane;

5) The filtered-through solution is acidified to pH value 2.5 by using 98% sulfuric acid, followed by incubation at 80° C. for 2 hours.

6) The precipitate formed at the step 5) The crude tridecanedicarboxylic acid is obtained by centrifuging the precipitate formed at the step 5). The precipitate is then re-suspended and washed by using water and then centrifuged again, followed by vacuum drying to obtain the crude tridecanedicarboxylic acid.

3. The pretreatment process before crystallization

1) The crude tridecanedicarboxylic acid is re-dissolved using 5M NaOH solution at a proportion of 1.65 g/ml, which is then heated to 80° C. and maintained for one hour. The solution containing re-dissolved tridecanedicarboxylic acid is then filtered to remove insoluble impurities with a piece of filter paper at 80° C. in a cabinet drier.

2) Active a carbon powder with a final amount of 0.2% (w/v) or a siliceous earth with 0.5% (w/v) is then added to the filtrate, which is is carried out at 60° C. and incubated at the temperature for 60 minutes.

3) Active carbon or siliceous earth powder is removed by vacuum filtration with a 0.44 μm nylon membrane;

4) The filtered-through solution is acidified to or lower than pH value 2.5 by using 98% sulfuric acid so that tridecanedicarboxylic acid is precipitated from the solution;

5) The deposition of tridecanedicarboxylic acid is collected by centrifuging;

6) The obtained tridecanedicarboxylic acid is then re-suspended and washed using water with three-fold of the acid mass, followed by centrifuging. This washing step is repeated till the pH value of the washing supernatant is higher than 6.5. This washing step is usually carried out for three times. Water with mass of five-folds of that of the dicarboxylic acid is added and the suspension is heated to 80° C., followed by filtering to get the filtered cake. The filtering cake is then washed for three times using water at 85° C.

4. The crystallization process of the tridecanedicarboxylic acid.

1) The filtering cake of the washed tridecanedicarboxylic acid is re-suspended in water with mass of five-folds of that of the filtering cake. The mixture is then heated to 118˜120° C. using a pressure vessel(GSHA-2 (2L), Henghua Chemical Plant, Weihai, P.R. China), and maintained the temperature for 20-30 minutes.

2) The temperature of the mixture is dropped continuously and slowly to the room temperature at a rate of 10° C. per hour. Tridecanedicarboxylic acid will be crystallized during the temperature dropping process;

3) The crystallized tridecanedicarboxylic acid is obtained by centrifuging. Some float on the aqueous surface of the mixture, which contains monocarboxylic acid, is separated and removed using this method;

4) The residual moisture is dried in a vacuum oven; and the purified tridecanedicarboxylic acid is procured.

The obtained tridecanedicarboxylic acid is 124.4 g in total, and the purity is 98.8%, the yield of product is 92.8%.

### EXAMPLE 3

1. Culturing, seedling and fermentation of the strain for hexadecanedicarboxylic acid production

The strain used in the embodiment of the present invention is Candida tropicalis UH-2-48(deposit number CGMCC 0239). A 50 ml starter culture is prepared from a conventional slant, and cultivated for 16 hours, followed by being transferred to a 1L further secondary starter culture for another 16 hours. The starter culture contains corn syrup 0.2˜0.5% (w/v), yeast extract 0.3˜0.7% (w/ v), urea 0.2˜0.5% (w/v), sucrose 2˜5% (w/v), KH₂PO₄ 0.5˜1% (w/v), defoamer 0.03˜0.05% (v/v).

The aforementioned secondary starter culture is finally transferred to a 10L fermenter tank to carry out the final fermentation. The basic media of the culture with a volume of 4L contains corn syrup 2˜7% (w/v), NaCl 0.1%˜0.3% (w/v), yeast extract 0.15˜0.3% (w/v), urea 0.1˜0.25% (w/v), glucose 3˜7% (w/v), sucrose 0.5˜2% (w/v), KNO3 1˜2% (w/v), KH2PO4 0.5˜2% (w/v), cell regulators: 3˜7% (w/v), emulsifier 0.001˜0.05% (v/v), defoamer 0.03% (v/v). The fermentation is carried out for 144-156 hours at 30° C. with a ventilation volume at 1:0.5. Sodium palmitate is supplemented at the rate of 50 ml/h after the fermentation for 16 hours till 0.5M of sodium palmitate (approximately 2L) is thoroughly supplemented. The pH value is controlled at 7.2 with by 10M NaOH is solution, and the dissolved oxygen is maintained at 20% through adjusting the rotational speed.

2. Procurement of the crude tridecanedicarboxylic acid

1) A 7L fermentation liquid, which is obtained in the above fermentation procedures with a final concentration of the total acid content at 248.5 g/l, is heated to 80° C. and maintained for 60 minutes.

2) After pH value of NaOH (10M) is adjusted to 9.5, the yeast cells are removed by centrifuging.

3) Active carbon powder at final amount of 0.5% (w/v) is added to the supernatant and the filtrate is incubated at 70° C., which is maintained for 60 minutes.

4) Active carbon is removed by vacuum filtration with a 0.44 μm nylon membrane;

5) The filtered-through solution is acidified to pH 2.5 value by using 98% sulfuric acid, followed by incubation at 80° C. for 2 hours.

6)The crude tridecanedicarboxylic acid is obtained by centrifuging the precipitate formed at the step 5). The precipitate is then re-suspended and washed by using water and then centrifuged again, followed by vacuum drying to obtain the crude tridecanedicarboxylic acid.

3. The pretreatment process before crystallization

1) The crude tridecanedicarboxylic acid is re-dissolved by using 5M NaOH solution at a proportion of 1.40 g/ml, which is then heated to 80° C. and maintained for one hour. The solution containing re-dissolved tridecanedicarboxylic acid is then filtered to remove insoluble impurities with a piece of filter paper at 80° C. in a cabinet drier.

2) Active carbon powder at a final amount of 0.2% (w/v) or siliceous earth at 0.5% (w/v) is then added to the filtrate, which is carried out at 60° C. and incubated at the temperature for 60 minutes.

3) Active carbon or siliceous earth powder is removed by vacuum filtration with a 0.44 μm nylon membrane;

4) The filtered-through solution is acidified to or lower than pH 2.5 value by using 98% sulfuric acid so that tridecanedicarboxylic acid is precipitated from the solution;

5) The deposit of tridecanedicarboxylic acid is collected by centrifuging;

6) The obtained tridecanedicarboxylic acid is then re-suspended and washed by using water with mass of three-folds of that of the acid, followed by centrifuging. This washing step is repeated till the pH value of the washing supernatant is higher than 6.5. This washing step is usually carried out for three times. Water with mass of five-folds of that of the acid mass is added and the suspension is heated to 80° C., followed by filtration to get the filtered cake. The cake is then washed for three times using water at 85° C.

4. The crystallization process of the tridecanedicarboxylic acid.

1) The filtering cake of the washed tridecanedicarboxylic acid is re-suspended in water with mass of five-folds of that of the cake mass. The mixture is then heated to 124˜126 ° C. using a pressure vessel(GSHA-2 (2L), Henghua Chemical Plant, Weihai, P.R. China), and maintained at the temperature for 20-30 minutes.

2) The temperature of the mixture is dropped continuously and slowly to the room temperature at a rate of 10° C. per hour. Tridecanedicarboxylic acid will be crystallized during the temperature dropping process;

3) The crystallized tridecanedicarboxylic acid is obtained by centrifuging. Some float on the aqueous surface of the mixture, which contains monocarboxylic acid, is separated and removed using this method;

4) The residual moisture is dried in a vacuum oven; and the purified tridecanedicarboxylic acid is procured.

The obtained tridecanedicarboxylic acid is 218.2 g in total, and the purity is 98.8%, the yield of product is 87.8%.

### EXAMPLE 4

The method of the example 3 is same with the method of example 4, the difference is only that the raw material is 160 g methyl hexadecanoate, and the final product is 121.5 g tridecanedicarboxylic acid, and the purity is 98.6%, the yield of product is 91.68%.

The properties of the product of example 1-4 are shown in Table 2. And the test of purity is the method of high performance liquid chromatography (shown in FIGS. 2a and 2b).

TABLE 2

| | | | sodium | |
|---|---|---|---|---|
| | | | palmitate | methyl |
| | nC₁₂ | nC₁₃ | (0.5M) | hexadecanoate |
| MW | 170 | 184 | 278 | 270 |
| Raw material (g) | 150 | 150 | 278 | 160 |
| Mass of pure diacid (g) | 138.2 | 124.1 | 218.2 | 121.5 |
| Mass of total acid^{a} (g/L) | 187 | 177 | 248.5 | 132.7 |
| Rate of raw material | 68.1 | 62.4 | 76.3 | 71.7 |
| conversion^{b} (%) | | | | |
| Yield of product (%)^{c} | 95.2 | 92.8 | 87.8 | 91.6 |
| Purity of the rude product | 87 | 87 | 83 | 85 |
| (%)^{d} | | | | |
| Purity of the crystallization | 99.2 | 98.8 | 98.6 | 98.6 |
| product^{d} (%) | | | | |

^{a}Mass of total acid, which is tested using the titration method with NaOH (CN 1130685, example 1);
^{b}Mol conversion rate, i.e. the mol of the final product diacid/the mol of the raw material × 100%;
^{c}the mass of the total acid/the mass of the acid in the fermentation supernatant × 100%;
^{d}the test method is high performance liquid chromatography.

What is claimed is:

1. A process for purification or refinery of a long-chain dicarboxylic acid or a salt thereof comprising the steps of:
1). Acidifying a crude material which contains a long-chain dicarboxylic acid or a salt thereof and collecting a precipitated crude product of a dicarboxylic acid which is in solid state, then processing step 2);
2). Dissolving the aforementioned crude product using an alkaline solution with heating to obtain a solution, filtering the solution to remove an insoluble material in the solution and decoloring the solution with a decoloring agent to obtain a filtered solution;
3). Acidifying the filtered solution to adjust a first pH value to 1˜2.5, and collecting a deposit of the dicarboxylic acid after centrifuging the filtered solution;
4). Washing the deposit of the dicarboxylic acid to neutrality by adding water with a mass of 3 to 20 folds of that of the dicarboxylic acid to re-suspend the dicarboxylic acid, heating the deposit of the dicarboxylic acid up to 60˜100° C., filtering, washing, and drying the deposit of the dicarboxylic acid to get a filtration cake of the dicarboxylic acid;
5). Suspending the filtration cake of the dicarboxylic acid in water, heating a mixture of the filtration cake of the dicarboxylic acid and water to a first temperature above 100° C. under a high pressure, and maintaining the first temperature above a melting point of the dicarboxylic acid for 20˜30 minutes;
6) Slowly dropping the first temperature of the mixture of the filtration cake of the dicarboxylic acid and water to a room temperature and procuring the dicarboxylic acid crystals by filtering.

2. The process according to claim 1, wherein in the process 1), a second pH value during the step of acidifying and depositing the filtrate is 1˜2.5, and a second temperature during the step of acidifying and depositing the filtrate is 60˜100° C.

3. The process according to claim 1, wherein in the process 2), alkaline solution of 5M NaOH is used to heat and dissolve the dicarboxylic acid, and the condition for processing is at 80° C. for one hour.

4. The process according to claim 1, wherein in the process 2), a filter medium includes one kind or several kinds of gauze, nylon membrane, ceramic film, metal film or glass fiber membran.

5. The process according to claim 1, wherein in the process 2), the decoloring agent which is used for decoloring is an activated carbon with 0.2% w or a siliceous earth with 0.5% w.

6. The process according to claim 1, wherein in the process 4), the is deposit of the dicarboxylic acid is washed till a third pH value of the deposit of the dicarboxylic acid is 6.5˜7.0.

7. The process according to claim 1, wherein in the process 4), the mass of said water used in washing the deposit of the dicarboxylic acid is 5 folds to that of the dicarboxylic acid.

8. The process according to claim 1, wherein in the process 4), said water used in washing the deposit of the dicarboxylic acid is at 85° C. and with neutrality pH, and the deposit of the dicarboxylic acid is washed 1-10 times repeatedly.

9. The process according to claim 1, wherein in the process 5), said first temperature is the melting point of the dicarboxylic acid, and the duration of the heating is 10-60minutes.

10. The process according to claim 1, wherein in the process 6), said slowly dropping the first temperature is to drop the first temperature 10˜15° C. per hour.

11. A dicarboxylic acid produced by using the process of anyone of the claims 1-10, wherein no organic solvent is used in the purification process.

12. A process for purification or refinery of a long-chain dicarboxylic acid or a salt thereof comprising the steps of:
1). Removing an organic solvent component of an organic solvent solution which contains a long-dicarboxylic acid and/or a salt thereof, acidifying and collecting a precipitated crude product of a dicarboxylic acid which is in solid state, then processing step 2);
2). Dissolving the aforementioned crude product using an alkaline solution with heating to obtain a solution, filtering the solution to remove an insoluble material in the solution and decoloring the solution with a decoloring agent to obtain a filtered solution;
3). Acidifying the filtered solution to adjust a pH value to 1-2.5, and is collecting a deposit of the dicarboxylic acid after centrifuging the filtered solution;
4). Washing the deposit of the dicarboxylic acid to neutrality by adding water with a mass of 3 to 20 folds of that of the dicarboxylic acid to re-suspend the dicarboxylic acid, heating the deposit of the dicarboxylic acid up to 60˜100° C., filtering, washing, and drying the deposit of the dicarboxylic acid to get a filtration cake of the dicarboxylic acid;
5). Suspending the filtration cake of the dicarboxylic acid in water, heating a mixture of the filtration cake of the dicarboxylic acid and water to a temperature above 100° C. under a high pressure, and maintaining the temperature above a melting point of the dicarboxylic acid for 20˜30 minutes;
6) Slowly dropping the temperature of the mixture of the filtration cake of the dicarboxylic acid and water to a room temperature and procuring the dicarboxylic acid crystals by filtering.

13. A process for purification or refinery of a long-chain dicarboxylic acid or a salt thereof comprising the steps of:
1). Dissolving a solid crude product which contains a dicarboxylic acid and/or a salt thereof using an alkaline solution with heating to obtain a solution, filtering the solution to remove an insoluble material in the solution and discoloring with a decoloring agent to obtain a filtered solution;
2). Acidifying the filtered solution to adjust a pH value to 1˜2.5, and collecting a deposit of the dicarboxylic acid after centrifuging the filtered solution;
3). Washing the deposit of the dicarboxylic acid to neutrality by adding water with a mass of 3 to 20 folds of that of the dicarboxylic acid to re-suspend the dicarboxylic acid, heating the deposit of the dicarboxylic acid up to 60˜100° C., filtering, washing, and drying the deposit of the dicarboxylic acid to get a filtration cake of the dicarboxylic acid;
4). Suspending the filtration cake of the dicarboxylic acid in water, heating a mixture of the filtration cake of the dicarboxylic acid and water to a temperature above 100° C. under a high pressure, and maintaining the temperature above a melting point of the dicarboxylic acid for 20˜30 minutes;
5) Slowly dropping the temperature of the filtration cake of the dicarboxylic acid and water to a room temperature and procuring the dicarboxylic acid crystals by filtering.
